(19) United States
(12) Patent Application Publication
(10) Pub. No.: US 2003/0187037 A1
(43) Pub. Date: Oct. 2, 2003

(54) Acetamide derivatives and the use thereof as inhibitors of coagulation factors xa and viia

(76) Inventors: Werner Mederski, Erzhausen (DE); Horst Juraszyk, Seeheim-Jugenheim (DE); Dieter Dorsch, Ober-Ramstadt (DE); Christos Tsaklakidis, Weinheim (DE); Johannes Gleitz, Darmstadt (DE); Christopher Barnes, Bad Soden (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/343,196

(22) PCT Filed: Jul. 3, 2001
(86) PCT No.: PCT/EP01/07594

(30) Foreign Application Priority Data
Jul. 29, 2000 (DE) 100 37 146.9

(51) Int. Cl. A61K 31/4245; A61K 31/325; A61K 31/277; A61K 31/195; A61K 31/165; C07D 271/02; C07C 327/26; C07C 279/10; C07C 271/50; C07C 255/42

(52) U.S. Cl. 514/364; 514/484; 514/513; 514/522; 514/534; 514/620; 548/125; 558/254; 558/414; 560/24; 560/34; 560/41; 564/147; 514/473; 549/229

(57) ABSTRACT

Novel compounds of the formula I, in which R, R¹ and R² are as defined in patent claim 1, are inhibitors of coagulation factor Xa and VIIa and can be employed for the treatment of thromboses, myocardial infarction, arteriosclerosis, inflammation, apoplexis, angina pectoris, restenosis after angioplasty, claudicatio intermittens, tumours, tumour diseases and/or tumour metastases.

The invention relates to compounds of the formula I

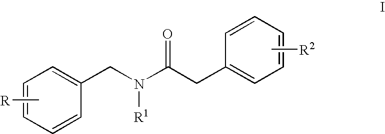

in which

R is CH₂NH₂, —CO—N═C(NH₂)₂, —NH—C(═NH)—NH₂ or —C(═NH)—NH₂, each of which may also be monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′-R³, COOA, COSA, COOAr, COOAr′ or by a conventional amino-protecting group, or is

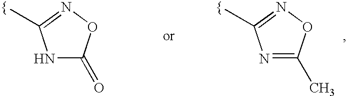

R¹ is unbranched, branched or cyclic alkyl having 1-20 carbon atoms, in which one or two CH₂ groups can be replaced by O or S atoms, or is Ar, Ar′ or X,

R² is phenyl which is monosubstituted by S(O)ₚA, S(O)ₚNHA, CF₃, COOA, CH₂NHA, CN or OA,

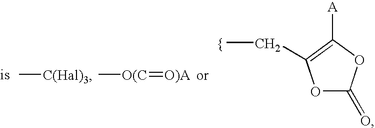

R³

Ar is phenyl or naphthyl, each of which is unsubstituted or mono-substituted, disubstituted or trisubstituted by A, OA, NAA′, NO₂, CF₃, CN, Hal, NHCOA, COOA, CONAA′, S(O)ₚA or S(O)ₚNAA′,

Ar′ is —(CH₂)ₙ—Ar,

A is H or unbranched, branched or cyclic alkyl having 1-20 carbon atoms,

A′ is unbranched, branched or cyclic alkyl having 1-10 carbon atoms,

Het is a monocyclic or bicyclic saturated, unsaturated or aromatic heterocyclic radical having from 1 to 4 N, O and/or S atoms, bonded via N or C, which may be unsubstituted or substituted by A,

X is —(CH₂)ₙ—Y,

Y is COOA or

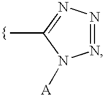

Hal is F, Cl, Br or I,

m is 0 or 1,

n is 1, 2, 3, 4, 5 or 6,

p is 0, 1 or 2,

and pharmaceutically tolerated salts, solvates and stereoisomers thereof.

The invention also relates to the optically active forms, the racemates, the diastereomers and the hydrates and solvates, for example alcoholates, of these compounds.

The invention had the object of finding novel compounds having valuable properties, in particular those which can be used for the preparation of medicaments.

It has been found that the compounds of the formula I and their salts have very valuable pharmacological properties while being well tolerated. In particular, they exhibit factor Xa-inhibiting properties and can therefore be employed for combating and preventing thromboembolic illnesses, such as thrombosis, myocardial infarction, arteriosclerosis, inflammation, apoplexia, angina pectoris, restenosis after angioplasty and claudicatio intermittens.

The compounds of the formula I according to the invention may furthermore be inhibitors of coagulation factor VIIa, factor IXa and thrombin in the blood coagulation cascade.

Aromatic amidine derivatives having an antithrombotic action are disclosed, for example, in EP 0 540 051 B1, WO 98/28269, WO 00/71508, WO 00/71511, WO 00/71493, WO 00/71507, WO 00/71509, WO 00/71512, WO 00/71515 or WO 00/71516. Cyclic guanidines for the treatment of thromboembolic illnesses are described, for example, in WO 97/08165. Aromatic heterocyclic compounds having factor Xa-inhibitory activity are disclosed, for example, in WO 96/10022. Substituted N-[(aminoiminomethyl)phenylalkyl]azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic illnesses. However, inhibition of thrombin can inhibit the fibrin formation involved in thrombus formation.

The inhibition of thrombin can be measured, for example, by the method of G. F. Cousins et al. in Circulation 1996, 94, 1705-1712.

Inhibition of factor Xa can thus prevent the formation of thrombin. The compounds of the formula I according to the invention and their salts engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example, by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273,12089-12094.

The compounds according to the invention can furthermore be used for the treatment of tumours, tumour diseases and/or tumour metastases. A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The compounds of the formula I can be employed as medicament active ingredients in human and veterinary medicine, in particular for the treatment and prevention of thromboembolic diseases, such as thrombosis, myocardial infarction, arteriosclerosis, inflammation, apoplexia, angina pectoris, restenosis after angioplasty, claudicatio intermittens, venous thrombosis, pulmonary embolism, arterial thrombosis, myocardial ischaemia, unstable angina and thrombosis-based strokes.

The compounds according to the invention are also employed for the treatment or prophylaxis of atherosclerotic diseases, such as coronary arterial disease, cerebral arterial disease or peripheral arterial disease. The compounds are also employed in combination with other thrombolytics in myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis.

The compounds are furthermore used in the cleaning of catheters and medical aids in patients in vivo, or as anticoagulants for the preservation of blood, plasma and other blood products in vitro. The compounds according to the invention are furthermore used in diseases in which blood coagulation makes a crucial contribution to the course of the disease or represents a source of secondary pathology, for example in cancer, including metastasis, inflammatory diseases, including arthritis, and diabetes.

In the treatment of the diseases described, the compounds according to the invention are also employed in combination with other thrombolytically active compounds, for example with the tissue plasminogen activator t-PA, modified t-PA, streptokinase or urokinase. The compounds according to the invention are administered either simultaneously with or before or after the other substances mentioned.

Particular preference is given to simultaneous administration with aspirin in order to prevent recurrence of the formation of thrombi.

The compounds according to the invention are also used in combination with blood platelet glycoprotein receptor (IIb/IIIa) antagonists which inhibit blood platelet aggregation.

The invention relates to the compounds of the formula I and salts thereof and to a process for the preparation of compounds of the formula I according to claim 1 in which R is amidino, and salts thereof, characterised in that

a) they are liberated from one of their functional derivatives by treatment with a solvolysing or hydrogenolysing agent,

and/or

b) a base or acid of the formula I is converted into one of its salts.

For all radicals which occur more than once, their meanings are independent of one another.

The following abbreviations are used:

| | |
|---|---|
| Ac | acetyl |
| BOC | tert-butoxycarbonyl |
| CBZ or Z | benzyloxycarbonyl |
| DAPECI | N-(3-dimethylaminopropyl)-N-ethylcarbodiimide |
| DCCI | dicyclohexylcarbodiimide |
| DMF | dimethylformamide |
| Et | ethyl |
| Fmoc | 9-fluorenylmethoxycarbonyl |
| HOBt | 1-hydroxybenzotriazole |
| Me | methyl |
| HONSu | N-hydroxysuccinimide |
| OBut | tert-butyl ester |
| Oct | octanoyl |
| OMe | methyl ester |
| OEt | ethyl ester |
| RT | room temperature |
| THF | tetrahydrofuran |
| TFA | trifluoroacetic acid |
| Trt | trityl (triphenylmethyl). |

Above and below, the radicals and parameters R, R¹, R², R³, Ar, Ar′, A, A′, Het, X, Y, n, m and p have the meanings indicated for the formula I, unless expressly stated otherwise.

A is H or alkyl, where alkyl is unbranched (linear), branched or cyclic and has from 1 to 20, preferably 1, 2, 3, 4, 5, 6, 7, 8, 9 or 10 carbon atoms. A is preferably methyl, furthermore ethyl, propyl, isopropyl, butyl, isobutyl, sec-butyl or tert-butyl, further also pentyl, 1-, 2- or 3-methylbutyl, 1,1-, 1,2- or 2,2-dimethylpropyl, 1-ethylpropyl, hexyl, 1-, 2-, 3- or 4-methylpentyl,- 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A is very particularly preferably H or alkyl having 1-6 carbon atoms, preferably methyl, ethyl, propyl, isopropyl, butyl, isobutyl, sec-butyl, tert-butyl, pentyl or hexyl.

A is furthermore, for example, cyclopropyl, cyclobutyl, cyclopentyl, cyclohexyl or cyclohexylmethyl.

A′ is alkyl, where alkyl is unbranched (linear), branched or cyclic and has from 1 to 10, preferably 1, 2, 3, 4, 5, 6, 7 or 8 carbon atoms. A′ is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A′ is particularly preferably alkyl having 16 carbon atoms, preferably methyl, ethyl, propyl, isopropyl, butyl, isobutyl, sec-butyl, tert-butyl, pentyl or hexyl.

A′ is furthermore, for example, cyclopentyl or cyclohexyl.

A′ is very particularly preferably alkyl having 1-6 carbon atoms, preferably methyl, ethyl, propyl, isopropyl, butyl, isobutyl, sec-butyl, tert-butyl, pentyl or hexyl.

Cyclic alkyl or cycloalkyl is preferably cyclopropyl, cyclobutyl, cyclopentyl, cyclohexyl or cycloheptyl.

Hal is preferably F, Cl or Br, but also I.

Ar is phenyl or naphthyl, each of which is unsubstituted or monosubstituted, disubstituted or trisubstituted by A, OA, NAA′, NO₂, CF₃, CN, Hal, NHCOA, COOA, CONAA′, S(O)ₚA or S(O)ₚNAA′.

Preferred substituents for phenyl or naphthyl are, for example, methyl, ethyl, propyl, butyl, OH, methoxy, ethoxy, propoxy, butoxy, amino, methylamino, dimethylamino, ethylamino, diethylamino, nitro, trifluoromethyl, fluorine, chlorine, acetamido, methoxycarbonyl, ethoxycarbony, aminocarbonyl, sulfonamido, methylsulfonamido, ethyl sulfonamido, propylsulfonamido, butylsulfonamido, tert-butylsulfonamido, tert-butylaminosulfonyl, dimethylsulfonamido, phenylsulfonamido, carboxyl, dimethylaminocarbonyl, phenylaminocarbonyl, acetyl, propionyl, benzoyl, methylsulfonyl or phenylsulfonyl.

Ar is particularly preferably, for example, unsubstituted phenyl or phenyl which is monosubstituted by SO₂NH₂, SO₂CH₃, fluorine or alkoxy, for example methoxy.

Ar′ is —(CH₂)ₙ—Ar, preferably benzyl which is unsubstituted or monosubstituted, disubstituted or trisubstituted by fluorine and/or chlorine.

Y is preferably, for example, methoxycarbonyl, ethoxycarbonyl or 1-methyltetrazol-5-yl.

In X, n is preferably, for example, 1 or 2.

Het is preferably, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl.

The heterocyclic radicals can also be partially or completely hydrogenated.

Het can thus, for example, also be 2,3-dihydro-2-, -3-, -4- or -5-furyl, 2,5-dihydro-2-, -3-, 4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, further preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or alternatively 3,4-dihydro-2H-1,5-benzodioxepin-6- or -7-yl, furthermore preferably 2,3-dihydrobenzofuranyl or 2,3-dihydro-2-oxofuranyl.

Het is particularly preferably, for example, furyl, thienyl, thiazolyl, imidazolyl, 2,1,3-benzothiadiazolyl, oxazolyl, pyridyl, indolyl, 1-methylpiperidinyl, piperidinyl or pyrrolidinyl, very particularly preferably pyridyl, 1-methylpiperidin-4-yl or piperidin-4-yl.

R is preferably, for example, amidino, N-methoxycarbonylamidino, N-ethoxycarbonylamidino, N-(2,2,2-trichloroethoxycarbonyl)amidino, N-ethylthiocarbonylamidino, N-benzyloxycarbonylamidino, N-phenoxycarbonylamidino, N-(4-fluorophenoxycarbonyl)amidino, N-(4-methoxyphenylthiocarbonyl)amidine, N—[CH₃CO—O—CH(CH₃)—O—CO]amidine=N-acetoxyethoxycarbonylamidine, N-ethoxycarbonyloxyamidine, N-(N,N-diethylaminoethoxycarbonyl)amidino, N-[(1-methylpiperidin-4-yl)oxycarbonyl]amidino or N-[(pyridin-2-yl)ethoxycarbonyl]amidino. R is preferably in the meta-position of the phenyl ring.

R¹ is preferably, for example, benzyl, methyl, ethyl, propyl, butyl, isopropyl, isobutyl, sec-butyl, pentyl, pent-3-yl, cyclohexylmethyl, 4-fluorobenzyl, ethoxycarbonylmethyl, ethoxycarbonylethyl, (1-methyltetrazol-5-yl)ethyl, methoxyethyl, methoxymethyl or methoxybutyl.

R² is preferably, for example, phenyl which is monosubstituted by SO₂NH₂ or SO₂Me.

The compounds of the formula I can have one or more centres of chirality and therefore occur in various stereoisomeric forms. The formula I covers all these forms.

Accordingly, the invention relates in particular to the compounds of the formula I in which at least one of the radicals mentioned has one of the preferred meanings indicated above. Some preferred groups of compounds can be expressed by the following sub-formulae Ia to Ii, which conform to the formula I and in which the radicals not designated in greater detail have the meaning indicated in the formula I, but in which

in Ia R is —C(═NH)—NH₂, which may also be monosubstituted by OH or a conventional amino-protecting group, or is

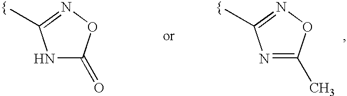

in Ib R is —C(═NH)—NH₂, which may also be monosubstituted by OH or a conventional amino-protecting group, or is

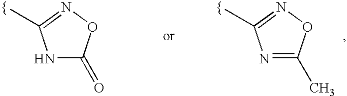

R¹ is unbranched, branched or cyclic alkyl having 1-8 carbon atoms, in which one CH₂ group may be replaced by O, or is Ar, Ar′ or X;

in Ic R is —C(═NH)—NH₂, which may also be monosubstituted by OH or a conventional amino-protecting group, or is

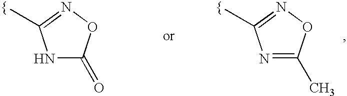

R¹ is unbranched, branched or cyciic alkyl having 1-8 carbon atoms, in which one CH₂ group may be replaced by O, or is Ar, Ar′ or X,

R² is phenyl which is mohosubstituted by SA, SOA, SO₂A, SO₂NHA, CF₃, COOA, CH₂NHA, CN or OA;

in Id R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(OH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

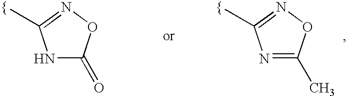

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A;

in Ie R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

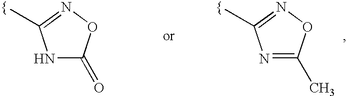

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂;

in If R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

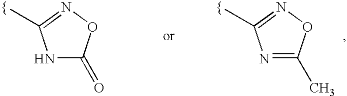

R¹ is unbranched, branched or cyclic aikyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine;

in Ig R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

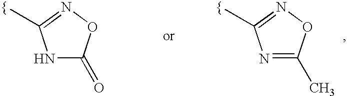

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

A and A′ are each, independently of one another, H or unbranched, branched or cyclic alkyl having 1-8 carbon atoms;

in Ih R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

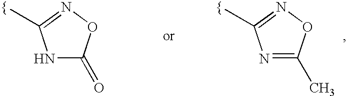

R¹ is unbranched, branched or cyclic aikyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

Het is a monocyclic saturated or aromatic heterocyclic radical having from 1 to 2 N and/or O atoms,

in Ii R is CH₂NH₂, CH₂NHCOA or CH₂NHCOOA, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is

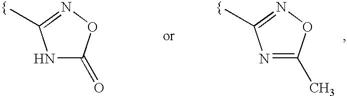

R¹ is unbranched, branched or cyclic alkyl having 1-8 carbon atoms, in which one CH₂ group may be replaced by O, or is Ar, Ar′ or X,

R² is phenyl which is monosubstituted by SA, SOA, SO₂A, SO₂NHA, CF₃, COOA, CH₂NHA, CN or OA,

R³ is —CCl₃ or —O(C═O)A,

Ar is phenyl which is unsubstituted or monosubstituted by A, OA, CF₃, Hal or SO₂NH₂,

Ar′ is benzyl which is unsubstituted or monosubstituted, disubstituted or trisubstituted by fluorine,

Het is a monocyclic saturated or aromatic heterocyclic radical having from 1 to 2 N and/or O atoms,

and pharmaceutically tolerated salts and solvates thereof.

The compounds of the formula I and also the starting materials for the preparation are, in addition, prepared by methods known per se, as described in the literature (for example, in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

Compounds of the formula I can preferably be obtained by liberating compounds of the formula I from one of their functional derivatives by treatment with a solvolysing or hydrogenolysing agent.

Preferred starting materials for the solvolysis or hydrogenolysis are those which conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ is an amino-protecting group, instead of an HN group, and/or those which carry an hydroxyl-protecting group instead of the H atom of an hydroxyl group, for example those which conform to the formula I, but carry a —COOR″ group, in which R″ is an hydroxyl-protecting group, instead of a —COOH group.

Preferred starting materials are also the oxadiazole derivatives which can be converted into the corresponding amidino compounds.

The liberation of the amidino group from its oxadiazole derivative can be carried out, for example, by treatment with hydrogen in the presence of a catalyst (for example water-moist Raney nickel). Suitable solvents are those indicated below, in particular alcohols, such as methanol or ethanol, organic acids, such as acetic acid or propionic acid, or mixtures thereof. The hydrogenolysis is generally carried out at temperatures between about 0 and 100° and pressures between about 1 and 200 bar, preferably at 20-30° (room temperature) and 1-10 bar.

The oxadiazole group is introduced, for example, by reaction of the cyano compounds with hydroxylamine and reaction with phosgene, dialkyl carbonate, chloroformates, N,N′-carbonyldiimidazole or acetic anhydride.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and toluyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The compounds of the formula I are liberated from their functional derivatives=13 depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 500, preferably between 15 and 30° (room temperature).

The BOC, OBut and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the-CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

An SO₂NH₂ group, for example in R², is preferably employed in the form of its tert-butyl derivative. The tert-butyl group is cleaved off, for example, using TFA with or without addition of an inert solvent, preferably with addition of a small amount of anisole (1-10% by volume).

A cyano group is converted into an amidino group by reaction with, for example, hydroxylamine followed by reduction of the N-hydroxyamidine using hydrogen in the presence of a catalyst, such as, for example, Pd/C. In order to prepare an amidine of the formula I (for example Ar=phenyl which is monosubstituted by C(═NH)—NH₂), it is also possible to add ammonia onto a nitrile. The adduction is preferably carried out in a multistep process by, in a manner known per se, a) converting the nitrile into a thioamide using H₂S, converting the thioamide into the corresponding S-alkylimidothioester using an alkylating agent, for example CH₃I, and in turn reacting the thioester with NH₃ to give the amidine, b) converting the nitrile into the corresponding imidoester using an alcohol, for example ethanol, in the presence of HCl, and treating this ester with ammonia, or c) reacting the nitrile with lithium bis(trimethylsilyl)amide, and subsequently hydrolysing the product.

The precursors of the compounds of the formula I are prepared, for example, by reacting compounds of the formula II

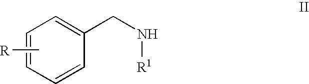

in which

R is CN, —CO—N═C(NH₂)₂, —NH—C(═NH)—NH₂ or —C(═NH)—NH₂ which is monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′—R³, COOA, COSA, COOAr, COOAr′ or by a conventional amino-protecting group,

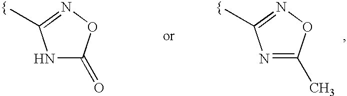

and R¹ is as defined in claim 1, with compounds of the formula III

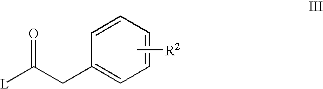

in which L is Cl, Br, I or a free or reactively functionally modified OH group, and R² is, for example, Br.

In the compounds of the formula II, L is preferably Cl, Br, I or a free or reactively modified OH group, such as, for example, an activated ester, an imidazolide or alkylsulfonyloxy having 1-6 carbon atoms (preferably methylsulfonyloxy) or arylsulfonyloxy having 6-10 carbon atoms (preferably phenyl- or p-tolylsulfonyloxy).

The reaction of the carboxylic acid derivatives of the formula III with the amine components of the formula II is carried out in a manner known per se, preferably in a protic or aprotic, polar or nonpolar inert organic solvent.

Some of the compounds of the formulae II or III used as intermediates are known or can be prepared by conventional methods.

However, a preferred variant also comprises reacting the reactants directly with one another, without addition of a solvent.

It is likewise advantageous to carry out the reactions described in the presence of a base or with an excess of the basic component. Examples are suitable solvents are preferably alkali metal or alkaline earth metal hydroxides, carbonates or alkoxides or organic bases, such as triethylamine or pyridine, which are also used in excess and can then simultaneously serve as solvent.

Suitable inert solvents are, in particular, alcohols, such as methanol, ethanol, isopropanol, n-butanol or tert-butanol; ethers, such as diethyl ether, diisopropyl ether, THF or dioxane; glycol ethers, such as ethylene glycol monomethyl or monoethyl ether or ethylene glycol dimethyl ether (diglyme); ketones, such as acetone or butanone; nitrites, such as acetonitrile; nitro compounds, such as nitromethane or nitrobenzene esters, such as ethyl acetate; amides, such as hexamethylphosphoric triamide; sulfoxides, such as dimethyl sulfoxide (DMSO); chlorinated hydrocarbons, such as dichloromethane, chloroform, trichloroethylene, 1,2-dichloroethane or carbon tetrachloride; or hydrocarbons, such as benzene, toluene or xylene. Also suitable are mixtures of these solvents with one another.

Particularly suitable solvents are methanol, THF, dimethoxyethane, dioxane, water or mixtures which can be prepared therefrom. Suitable reaction temperatures are, for example, temperatures between 20° and the boiling point of the solvent. The reaction times are between 5 minutes and 30 hours. It is advantageous to employ an acid scavenger in the reaction. Suitable for this purpose are all types of bases which do not interfere with the reaction itself. Particularly suitable, however, is the use of inorganic bases, such as potassium carbonate, or of organic bases, such as triethylamine or pyridine.

Esters can be saponified, for example, using acetic acid or using NaOH or KOH in water, water/THF or water/dioxane at temperatures between 0 and 100°.

The products obtained in the reaction of the compounds of the formula II with the compounds of the formula III are then reacted further with the appropriate boronic acid derivatives to give the biphenyl precursors, for example by reaction in a Suzuki reaction. The Suzuki reaction is advantageously carried out with palladium mediation, preferably by addition of Pd(PPh₃)₄ or PD(II)Cl₂dppf, in the presence of a base, such as potassium carbonate, in an inert solvent or solvent mixture, for example DMF, at temperatures between 0° and 150°, preferably between 60° and 120°. Depending on the conditions used, the reaction time is between a few minutes and a number of days. The boronic acid derivatives can be prepared by conventional methods or are commercially available. The reactions can be carried out analogously to the methods indicated in Suzuki et al., J. Am. Chem. Soc. 1989, 111, 314 ff. and in Suzuki et al. Chem. Rev. 1995, 95, 2457 ff.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or poly-basic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate). It is also possible to use physiologically acceptable organic bases, such as, for example, ethanolamine.

Compounds of the formula I according to the invention may be chiral, owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline) or N-benzenesulfonylproline), or the various optically active camphorsulfonic acids. Also advantage is chromatographic enantiomer resolution with the aid of an optically active resolving agent (for example dinitrobenzoylphenylglycine, cellulose triacetate or other derivatives of carbohydrates or chirally derivatized methacrylate polymers immobilized on silica gel). Examples of suitable eluents for this purpose are aqueous or alcoholic solvent mixtures, such as, for example, hexane/isopropanol/acetonitrile, for example in the ratio 82:15:3.

The invention furthermore relates to the use of compounds of the formula I and/or their physiologically acceptable salts for the preparation of pharmaceutical preparations, in particular by non-chemical methods. They can be converted here into a suitable dosage form together with at least one solid, liquid and/or semi-liquid excipient or assistant and, if desired, in combination with one or more further active ingredients.

The invention thus also relates to pharmaceutical preparations comprising at least one medicament according to one of claims 5 and 6 and, if desired, excipients and/or assistants and, if desired, other active ingredients.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or Vaseline. Suitable for oral administration are, inparticular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, to prepare injection preparations. The preparations indicated may be sterilized and/or comprise assistants, such as lubricants, preservatives, stabilizers and/or wetting agents, emulsifying agents, salts for modifying the osmotic pressure, buffer substances, colorants and flavours and/or a plurality of further active ingredients, for example one or more vitamins.

The invention also relates to the use of compounds according to claims 1 and 2 and/or their physiologically acceptable salts for the preparation of a medicament for combating thromboembolic illnesses, such as thrombosis, myocardial infarction, arteriosclerosis, inflammation, apoplexia, angina pectoris, restenosis after angioplasty and claudicatio intermittens.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Above and below, all temperatures are given in ° C. In the following examples, ‘conventional work-up’ means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallization. Rf values on silica gel; eluent: ethyl acetate/methanol 9:1.

Mass spectrometry (MS): EI (electron ionisation) M⁺ FAB (fast atom bombardment) (M+H)⁺

### EXAMPLE 1

Preparation of Starting Materials of the Formula II

Precursors from the N-Propyl Series

1.1

10.0 ml of triethylamine are added to a solution of 4.6 ml of n-propylamine in 100 ml of THF. 8.5 ml of trifluoroacetic anhydride are then added drop-wise. After stirring for 4 hours, the mixture is subjected to conventional work-up, giving 5.58 g of N-propyl-2,2,2-trifluoroacetamide (“AA”) as a yellow oil, EI 155.

1.2

13.0 g of caesium carbonate are added to a solution of 5.0 g of “AA” in 200 ml of DMF, and the mixture is stirred at RT for 0.5 hour. 10.0 g of 3-[3-bromomethyl)phenyl]-5-methyl-1,2,4-oxadiazole (“AB”) are then added dropwise, and the mixture is stirred for a further 18 hours. Conventional work-up gives 9.32 g of 2,2,2-trifluoro-N-propyl-N{3-[5-methyl(1,2,4-oxadiazol)-3-yl]benzyl}acetamide (“AC”) as a yellow oil, FAB 328.

1.3

1.9 g of lithium hydroxide and 15 ml of water are added to a solution of 8.5 g of “AC” in 300 ml of methanol, and the mixture is stirred for 2.5 hours. Conventional work-up gives 4.51 g of [3-(5-methyl-1,2,4-oxadiazol-3-yl)benzyl]propylamine (“AD”) as a yellow oil, FAB 232.

Precursors from the Phenyl Series

1.4

Analogously to Example 1.1, 5.0 ml of aniline give 10.25 g of N-phenyl-2,2,2-trifluoroacetamide (“BA”), FAB 190.

1.5

Analogously to Example 1.2, 6.0 g of “BA” give 9.37 g of 2,2,2-trifluoro-N-phenyl-N-{3-[5-methyl(1,2,4-oxadiazol)-3-yl]benzyl}acetamide (“BB”), FAB 362.

1.6

Analogously to Example 1.3, 9.5 g of “BB” give 6.61 g of [3-(5-methyl-1,2,4-oxadiazol-3-yl)benzyl]phenylamine (“BC”), m.p. 75-76°, FAB 266.

### EXAMPLE 2

2.1

A solution of 1.31 g of “AD”, 1.22 g of 4-bromophenylacetic acid, 1.09 g of N-(3-dimethylaminopropyl)-N′-ethylcarbodiimide hydrochloride, 0.76 g of 1-hydroxybenzotriazole and 0.62 ml of 4-methylmorpholine in 40 ml of DMF is stirred at RT for 6 hours. Conventional work-up gives 2.33 g of N-[3-(5-methyl-1,2,4-oxadiazol-3-yl)benzyl]-N-propyl-2-(4-bromophenyl)-acetamide (“AE”), EI 427/429.

2.2

Analogously to Example 2.1, 1.5 g of “BC” give 2.23 of N-[3-(5-methyl-1,2,4-oxadiazol-3-yl)benzyl]-N-phenyl-2-(4-bromophenyl)acetamide (“BD”), EI 427/429.

### EXAMPLE 3

3.1

1.5 g of 2-(tert-butylaminosulfonyl)phenylboronic acid, 12 ml of 2M sodium carbonate solution and 0.12 g of PdCl₂(dppf) are added successively to a solution of 1.0 g of “AE” in 60 ml of ethylene glycol dimethyl ether under an N₂ atmosphere, and the mixture is stirred at 85° for 2 hours. Conventional work-up gives 1.3 g of N-[3-(5-methyl-1,2,4-oxadiazol-3-yl)benzyl]-N-propyl-2-(2′-tert-butylsulfamoylbiphenyl-4-yl)acetamide (“CA”), m.p. 132-133°, FAB 561.

3.2

0.5 ml of acetic acid is added to a solution of 0.5 g of “CA” in 30 ml of methanol, and, after addition of 2.5 g of Raney nickel, the mixture is stirred under a hydrogen atmosphere for 18 hours. Removal of the catalyst and conventional work-up gives 0.46 g of N-3-amidinobenzyl-N-propyl-2-(2′-tert-butylsulfamoylbiphenyl-4-yl)acetamide (“CB”), FAB 521.

3.3

A solution of 0.35 g of “CB” in 3.5 ml of TFA and 0.35 ml of anisole is stirred at RT for 16 hours. Conventional work-up gives 0.26 g of N-3-amidinobenzyl-N-propyl-2-(2′-sulfamoylbiphenyl-4-yl)acetamide, FAB 465.

Affinity to Receptors:

| | | |
|---|---|---|
| IC₅₀ values [nM/litre] | IC₅₀ (factor Xa, human) | =2000.0 |
| | IC₅₀ (TF/VIIa) | =900.0 |

The following compounds are obtained analogously to Examples 1, 2 and 3.1-3.3

N-(3-amidinobenzyl)-N-methyl-2-(2′-sulfamoylbiphenyl-4-yl)acetamide,

N-(3-amidinobenzyl)-N-ethyl-2-(2′-sulfamoylbiphenyl-4-yl)acetamide,

N-(3-amidinobenzyl)-N-isopropyl-2-(2′-sulfamoylbiphenyl-4-yl)acetamide,

N-(3-amidinobenzyl)-N-butyl-2-(2′-sulfambylbiphenyl-4-yl)acetamide,

N-(3-amidinobenzyl)-N-isobutyl-2-(2-sulfamoylbiphenyl-4-yl)acetamide,

N-(3-amidinobenzyl)-N-pentyl-2-(2′-sulfamoylbiphenyl-4-yl)acetamide,

N-(3-amidinobenzyl)-N-sec-butyl-2-(2′-sulfamoylbiphenyl-4-yl)acetamide,

N-(3-amidinobenzyl)-N-cyclohexylmethyl-2-(2′-sulfamoylbiphenyl-4-yl)-acetamide,

N-(3-amidinobenzyl)-N-cyclohexyl-2-(2′-sulfamoylbiphenyl-4-yl)acetamide,

N-(3-amidinobenzyl)-N-cyclopentyl-2-(2′-sulfamoylbiphenyl-4-yl)acetamide,

N-(3-amidinobenzyl)-N-benzyl-2-(2′-sulfamoylbiphenyl-4-yl)acetamide,

N-(3-amidinobenzyl)-N-phenyl-2-(2′-sulfamoylbiphenyl-4-yl)acetamide, FAB 499.

Affinity to Receptors:

| | | |
|---|---|---|
| IC₅₀ values [nM/litre] | IC₅₀ (factor Xa, human) | =2000.0 |
| | IC₅₀ (TF/VIIa) | =1500.0 |

### EXAMPLE 4

4.1

Analogously to Example 3.1, 1.0 g of “AE” gives 1.0 g of N-[3-(5-methyl-1,2,4-oxadiazol-3-yl)benzyl]-N-propyl-2-(2′-methylsulfanylbiphenyl-4-yl)-acetamide (“DA”), EI 471.

4.2

0.9 g of “DA” and 1.5 g of sodium perborate trihydrate are suspended in 25 ml of acetic acid and stirred at RT for 48 hours. Conventional work-up gives 0.51 g of N-[3-(5-methyl-1,2,4-oxadiazol-3-yl)benzyl]-N-propyl-2-(2′-methylsulfonylbiphenyl-4-yl)acetamide (“DB”), EI 503.

4.3

Analogously to Example 3.2, 0.45 g of “DB” gives 0.37 g of N-(3-amidinobenzyl)-N-propyl-2-(2′-methylsulfonylbiphenyl-4-yl)acetamide, FAB 464.

| | | |
|---|---|---|
| IC₅₀ values [nM/litre] | IC₅₀ (factor Xa, human) | =1000.0 |
| | IC₅₀ (TF/VIIa) | =700.0 |

The following compounds are obtained analogously

N-(3-amidinobenzyl)-N-methyl-2-(2′-methylsulfonylbiphenyl-4-yl)acetamide,

N-(3-amidinobenzyl)-N-ethyl-2-(2′-methylsulfonylbiphenyl-4-yl)acetamide,

N-(3-amidinobenzyl)-N-isopropyl-2-(2′-methylsulfonylbiphenyl-4-yl)acetamide,

N-(3-amidinobenzyl)-N-butyl-2-(2′-methylsulfonylbiphenyl-4-yl)acetamide,

N-(3-amidinobenzyl)-N-isobutyl-2-(2′-methylsulfonylbiphenyl-4-yl)acetamide,

N-(3-amidinobenzyl)-N-pentyl-2-(2′-methylsulfonylbiphenyl-4-yl)acetamide,

N-(3-amidinobenzyl-N-sec-butyl-2-(2′-methylsulfonylbiphenyl-4-yl)acetamide,

N-(3-amidinobenzyl)-N-cyclohexylmethyl-2-(2′-methylsulfonylbiphenyl-4-yl)acetamide,

N-(3-amidinobenzyl)-N-cyclohexyl-2-(2′-methylsulfonylbiphenyl-4-yl)acetamide,

N-(3-amidinobenzyl)-N-cyclopentyl-2-(2′-methylsulfonylbiphenyl-4-yl)acetamide,

N-(3-amidinobenzyl)-N-benzyl-2-(2′-methylsulfonylbiphenyl-4-yl)acetamide,

N-(3-amidinobenzyl)-N-phenyl-2-(2′-methylsulfonylbiphenyl-4-yl)acetamide, FAB 498.

Affinity to Receptors:

| | | |
|---|---|---|
| IC₅₀ values [nM/litre] | IC₅₀ (factor Xa, human) | =550.0 |
| | IC₅₀ (TF/VIIa) | =650.0 |

### EXAMPLE 5

The reactions described in this example are carried out analogously to the method of S. M. Rahmathullah et al. in J. Med. Chem. 1999, 42, 3994-4000. The corresponding acid chlorides are first derivatised to give the 4-nitrophenylcarbonate compounds, which are then reacted further with the amidino compounds.

Starting from methyl chloroformate and reaction of the following “amidino compounds”

N-(3-amidinobenzyl)benzyl-N-propyl-2-(2′-sulfamoylbiphenyl-4-yl)acetamide,

N-(3-amidinobenzyl)-N-methyl-2-(2′-sulfamoyl biphenyl-4-yl)acetamide,

N-(3-amidinobenzyl)-N-ethyl-2-(2′-sulfamoylbiphenyl-4-yl)acetamide,

N-(3-amidinobenzyl)-N-isopropyl-2-(2′-sulfamoylbiphenyl-4-yl)acetamide,

N-(3-amidinobenzyl)-N-butyl-2-(2′-sulfamoylbiphenyl-4-yl)acetamide,

N-(3-amidinobenzyl)-N-isobutyl-2-(2′-sulfamoylbiphenyl-4-yl)acetamide,

N-(3-amidinobenzyl)-N-pentyl-2-(2′-sulfamoylbiphenyl-4-yl)acetamide,

N-(3-amidinobenzyl)-N-sec-butyl-2-(2′-sulfamoylbiphenyl-4-yl)acetamide,

N-(3-amidinobenzyl)-N-cyclohexylmethyl-2-(2′-sulfamoylbiphenyl-4-yl)acetamide,

N-(3-amidinobenzyl)-N-cyclohexyl-2-(2′-sulfamoylbiphenyl-4-yl)acetamide,

N-(3-amidinobenzyl)-N-cyclopentyl-2-(2-sulfamoylbiphenyl-4-yl)acetamide,

N-(3-amidinobenzyl)-N-benzyl-2-(2′-sulfamoylbiphenyl-4-yl)acetamide,

N-(3-amidinobenzyl)-N-phenyl-2-(2′-sulfamoylbiphenyl-4-yl)acetamide,

gives the following compounds

N-(3-N-methoxycarbonylamidinobenzyl)-N-propyl-2-(2′-sulfamoylbiphenyl-4-yl)acetamide,

N-(3-N-methoxycarbonylamidinobenzyl)-N-methyl-2-(2′-sulfamoylbiphenyl-4-yl)acetamide,

N-(3-N-methoxycarbonylamidinobenzyl)-N-ethyl-2-(2′-sulfamoylbiphenyl-4-yl)acetamide,

N-(3-N-methoxycarbonylamidinobenzyl)-N-isopropyl-2-(2′-sulfamoylbiphenyl-4-yl)acetamide,

N-(3-N-methoxycarbonylamidinobenzyl)-N-butyl-2-(2′-sulfamoylbiphenyl-4-yl)acetamide,

N-(3-N-methoxycarbonylamidinobenzyl)-N-isobutyl-2-(2′-sulfamoylbiphenyl-4-yl)acetamide,

N-(3-N-methoxycarbonylamidinobenzyl)-N-pentyl-2-(2′-sulfamoylbiphenyl-4-yl)acetamide,

N-(3-N-methoxycarbonylamidinobenzyl)-N-sec-butyl-2-(2′-sulfamoylbiphenyl-4-yl)acetamide,

N-(3-N-methoxycarbonylamidinobenzyl)-N-cyclohexylmethyl-2-(2′-sulfamoylbiphenyl-4-yl)acetamide,

N-(3-N-methoxycarbonylamidinobenzyl)-N-cyclohexyl-2-(2′-sulfamoylbiphenyl-4-yl)acetamide,

N-(3-N-methoxycarbonylamidinobenzyl)-N-cyclopentyl-2-(2′-sulfamoylbiphenyl-4-yl)acetamide,

N-(3-N-methoxycarbonylamidinobenzyl)-N-benzyl-2-(2′-sulfamoylbiphenyl-4-yl)acetamide,

N-(3-N-methxycarbonylamidinobenzyl)-N-phenyl-2-(2′-sulfamoylbiphenyl-4-yl)acetamide.

Starting from thioethyl chloroformate and reaction of the “amidino compounds” the following are held.

N-(3-N-ethylthiocarbonylamidinobenzyl)-N-propyl-2-(2′-sulfamoylbiphenyl-4-yl)acetamide,

N-(3-N-ethylthiocarbonylamidinobenzyl)-N-methyl-2-(2′-sulfamoylbiphenyl-4-yl)acetamide,

N-(3-N-ethylthiocarbonylamidinobenzyl)-N-ethyl-2-(2′-sulfamoylbiphenyl-4-yl)acetamide,

N-(3-N-ethylthiocarbonylamidinobenzyl)-N-isopropyl-2-(2′-sulfamoylbiphenyl-4-yl)acetamide,

N-(3-N-ethylthiocarbonylamidinobenzyl)-N-butyl-2-(2′-sulfamoylbiphenyl-4-yl)acetamide,

N-(3-N-ethylthiocarbonylamidinobenzyl)-N-isobutyl-2-(2′-sulfamoylbiphenyl-4-yl)acetamide,

N-(3-N-ethylthiocarbonylamidinobenzyl)-N-pentyl-2-(2′-sulfamoylbiphenyl-4-yl)acetamide,

N-(3-N-ethylthiocarbonylamidinobenzyl)-N-sec-butyl-2-(2′-sulfamoylbiphenyl-4-yl)acetamide,

N-(3-N-ethylthiocarbonylamidinobenzyl)-N-cyclohexylmethyl-2-(2′-sulfamoylbiphenyl-4-yl)acetamide,

N-(3-N-ethylthiocarbonylamidinobenzyl)-N-cyclohexyl-2-(2′-sulfamoylbiphenyl-4-yl)acetamide,

N-(3-N-ethylthiocarbonylamidinobenzyl)-N-cyclopentyl-2-(2′-sulfamoylbiphenyl-4-yl)acetamide,

N-(3-N-ethylthiocarbonylamidinobenzyl)-N-benzyl-2-(2′-sulfamoylbiphenyl-4-yl)acetamide,

N-(3-N-ethylthiocarbonylamidinobenzyl)-N-phenyl-2-(2′-sulfamoylbiphenyl-4-yl)acetamide.

Starting from 2,2,2-trichioroethyl chloroformate and reaction of the “amidino compounds” gives the following

N-(3-N-(2,2,2-trichloroethoxycarbonyl)amidinobenzyl)-N-propyl-2-(2′-sulfamoylbiphenyl-4-yl)acetamide,

N-(3-N-(2,2,2-trichloroethoxycarbonyl)amidinobenzyl)-N-methyl-2-(2′-sulfamoylbiphenyl-4-yl)acetamide,

N-(3-N-(2,2,2-trichloroethoxycarbonyl)amidinobenzyl)-N-ethyl-2-(2′-sulfamoylbiphenyl-4-yl)acetamide,

N-(3-N-(2,2,2-trichloroethoxycarbonyl)amidinobenzyl)-N-isopropyl-2-(2′-sulfamoylbiphenyl-4-yl)acetamide,

N-(3-N-(2,2,2-trichloroethoxycarbonyl)amidinobenzyl)-N-butyl-2-(2′-sulfamoylbiphenyl-4-yl)acetamide,

N-(3-N-(2,2,2-trichloroethoxycarbonyl)amidinobenzyl)-N-isobutyl-2-(2′-sulfamoylbiphenyl-4-yl)acetamide,

N-(3-N-(2,2,2-trichloroethoxycarbonyl)amidinobenzyl)-N-pentyl-2-(2′-sulfamoylbiphenyl-4-yl)acetamide,

N-(3-N-(2,2,2-trichloroethoxycarbonyl)amidinobenzyl)-N-sec-butyl-2-(2′-sulfamoylbiphenyl-4-yl)acetamide,

N-(3-N-(2,2,2-trichloroethoxycarbonyl)amidinobenzyl)-N-cyclohexylmethyl-2-(2′-sulfamoylbiphenyl-4-yl)acetamide,

N-(3-N-(2,2,2-trichloroethoxycarbonyl)amidinobenzyl)-N-cyclohexyl-2-(2′-sulfamoylbiphenyl-4-yl)acetamide,

N-(3-N-(2,2,2-trichloroethoxycarbonyl)amidinobenzyl)-N-cyclopentyl-2-(2′-sulfamoylbiphenyl-4-yl)acetamide,

N-(3-N-(2,2,2-trichloroethoxycarbonyl)amidinobenzyl)-N-benzyl-2-(2′-sulfamoylbiphenyl-4-yl)acetamide,

N-(3-N-(2,2,2-trichloroethoxycarbonyl)amidinobenzyl)-N-phenyl-2-(2′-sulfamoylbiphenyl4-yl)acetamide.

Starting from benzyl chloroformate and reaction of the “amidino compounds” gives the following

N-(3-N-benzyloxycarbonylamidinobenzyl)-N-propyl-2-(2′-sulfamoylbiphenyl-4-yl)acetamide,

N-(3-N-benzyloxycarbonylamidinobenzyl)-N-methyl-2-(2′-sulfamoylbiphenyl-4-yl)acetamide,

N-(3-N-benzyloxycarbonylamidinobenzyl)-N-ethyl-2-(2′-sulfamoylbiphenyl-4-yl)acetamide,

N-(3-N-benzyloxycarbonylamidinobenzyl)-N-isopropyl-2-(2′-sulfamoylbiphenyl-4-yl)acetamide,

N-(3-N-benzyloxycarbonylamidinobenzyl)-N-butyl-2-(2′-sulfamoylbiphenyl-4-yl)acetamide,

N-(3-N-benzyloxycarbonylamidinobenzyl)-N-isobutyl-2-(2′-sulfamoylbiphenyl-4-yl)acetamide,

N-(3-N-benzyloxycarbonylamidinobenzyl)-N-pentyl-2-(2′-sulfamoylbiphenyl-4-yl)acetamide,

N-(3-N-benzyloxycarbonylamidinobenzyl)-N-sec-butyl-2-(2′-sulfamoylbiphenyl-4-yl)acetamide,

N-(3-N-benzyloxycarbonylamidinobenzyl)-N-cyclohexylmethyl-2-(2′-sulfamoylbiphenyl-4-yl)acetamide,

N-(3-N-benzyloxycarbonylamidinobenzyl)-N-cyclohexyl-2-(2′-sulfamoylbiphenyl-4-yl)acetamide,

N-(3-N-benzyloxycarbonylamidinobenzyl)-N-cyclopentyl-2-(2′-sulfamoylbiphenyl-4-yl)acetamide,

N-(3-N-benzyloxycarbonylamidinobenzyl)-N-benzyl-2-(2′-sulfamoylbiphenyl-4-yl)acetamide,

N-(3-N-benzyloxycarbonylamidinobenzyl)-N-phenyl-2-(2′-sulfamoylbiphenyl-4-yl)acetamide.

Starting from phenyl chloroformate and reaction of the “amidino compounds” gives the following

N-(3-N-phenoxycarbonylamidinobenzyl)-N-propyl-2-(2′-sulfamoylbiphenyl-4-yl)acetamide,

N-(3-N-phenoxycarbonylamidinobenzyl)-N-methyl-2-(2′-sulfamoylbiphenyl-4-yl)acetamide,

N-(3-N-phenoxycarbonylamidinobenzyl)-N-ethyl-2-(2′-sulfamoylbiphenyl-4-yl)acetamide,

N-(3-N-phenoxycarbonylamidinobenzyl)-N-isopropyl-2-(2′-sulfamoylbiphenyl-4-yl)acetamide,

N-(3-N-phenoxycarbonylamidinobenzyl)-N-butyl-2-(2′-sulfamoylbiphenyl-4-yl)acetamide,

N-(3-N-phenoxycarbonylamidinobenzyl)-N-isobutyl-2-(2′-sulfamoylbiphenyl-4-yl)acetamide,

N-(3-N-phenoxycarbonylamidinobenzyl)-N-pentyl-2-(2′-sulfamoylbiphenyl-4-yl)acetamide,

N-(3-N-phenoxycarbonylamidinobenzyl)-N-sec-butyl-2-(2′-sulfamoylbiphenyl-4-yl)acetamide,

N-(3-N-phenoxycarbonylamidinobenzyl)-N-cyclohexylmethyl-2-(2′-sulfamoylbiphenyl-4-yl)acetamide,

N-(3-N-phenoxycarbonylamidinobenzyl)-N-cyclohexyl-2-(2′-sulfamoylbiphenyl-4-yl)acetamide,

N-(3-N-phenoxycarbonylamidinobenzyl)-N-cyclopentyl-2-(2′-sulfamoylbiphenyl-4-yl)acetamide,

N-(3-N-phenoxycarbonylamidinobenzyl)-N-benzyl-2-(2′-sulfamoylbiphenyl-4-yl)acetamide,

N-(3-N-phenoxycarbonylamidinobenzyl)-N-phenyl-2-(2′-sulfamoylbiphenyl-4-yl)acetamide.

Starting from 4-fluorophenyl chloroformate and reaction of the “amidino compounds” gives the following

N-(3-N-(4-fluorophenoxycarbonyl)amidinobenzyl)-N-propyl-2-(2′-sulfamoylbiphenyl-4-yl)acetamide,

N-(3-N-(4-fluorophenoxycarbonyl)amidinobenzyl)-N-methyl-2-(2′-sulfamoylbiphenyl-4-yl)acetamide,

N-(3-N-(4-fluorophenoxycarbonyl)amidinobenzyl)-N-ethyl-2-(2′-sulfamoylbiphenyl-4-yl)acetamide,

N-(3-N-(4-fluorophenoxycarbonyl)amidinobenzyl)-N-isopropyl-2-(2′-sulfamoylbiphenyl-4-yl)acetamide,

N-(3-N-(4-fluorophenoxycarbonyl)amidinobenzyl)-N-butyl-2-(2′-sulfamoylbiphenyl-4-yl)acetamide,

N-(3-N-(4-fluorophenoxycarbonyl)amidinobenzyl)-N-isobutyl-2-(2′-sulfamoylbiphenyl-4-yl)acetamide,

N-(3-N-(4-fluorophenoxycarbonyl)amidinobenzyl)-N-pentyl-2-(2′-sulfamoylbiphenyl-4-yl)acetamide,

N-(3-N-(4-fluorophenoxycarbonyl)amidinobenzyl)-N-sec-butyl-2-(2′-sulfamoylbiphenyl-4-yl)acetamide,

N-(3-N-(4-fluorophenoxycarbonyl)amidinobenzyl)-N-cyclohexylmethyl-2-(2′-sulfamoylbiphenyl-4-yl)acetamide,

N-(3-N-(4-fluorophenoxycarbonyl)amidinobenzyl)-N-cyclohexyl-2-(2′-sulfamoylbiphenyl-4-yl)acetamide,

N-(3-N-(4-fluorophenoxycarbonyl)amidinobenzyl)-N-cyclopentyl-2-(2′-sulfamoylbiphenyl-4-yl)acetamide,

N-(3-N-(4-fluorophenoxycarbonyl)amidinobenzyl)-N-benzyl-2-(2′-sulfamoylbiphenyl-4-yl)acetamide,

N-(3-N-(4-fluorophenoxycarbonyl)amidinobenzyl)-N-phenyl-2-(2′-sulfamoylbiphenyl-4-yl)acetamide.

Starting from thio-4-methoxyphenyl chloroformate and reaction of the “amidino compounds” gives the following

N-(3-N-(4-methoxyphenylthiocarbonyl)amidinobenzyl)-N-propyl-2-(2′-sulfamoylbiphenyl-4-yl)acetamide,

N-(3-N-(4-methoxyphenylthiocarbonyl)amidinobenzyl)-N-methyl-2-(2′-sulfamoylbiphenyl-4-yl)acetamide,

N-(3-N-(4-methoxyphenylthiocarbonyl)amidinobenzyl)-N-ethyl-2-(2′-sulfamoylbiphenyl-4-yl)acetamide,

N-(3-N-(4-methoxyphenylthiocarbonyl)amidinobenzyl)-N-isopropyl-2-(2′-sulfamoylbiphenyl-4-yl)acetamide,

N-(3-N-(4-methoxyphenylthiocarbonyl)amidinobenzyl)-N-butyl-2-(2′-sulfamoylbiphenyl-4-yl)acetamide,

N-(3-N-(4-methoxyphenylthiocarbonyl)amidinobenzyl)-N-isobutyl-2-(2′-sulfamoylbiphenyl-4-yl)acetamide,

N-(3-N-(4-methoxyphenylthiocarbonyl)amidinobenzyl)-N-pentyl-2-(2′-sulfamoylbiphenyl-4-yl)acetamide,

N-(3-N-(4-methoxyphenylthiocarbonyl)amidinobenzyl)-N-sec-butyl-2-(2′-sulfamoylbiphenyl-4-yl)acetamide

N-(3-N-(4-methoxyphenylthiocarbonyl)amidinobenzyl)-N-cyclohexylmethyl-2-(2′-sulfamoylbiphenyl-4-yl)acetamide,

N-(3-N-(4-methoxyphenylthiocarbonyl)amidinobenzyl)-N-cyclohexyl-2-(2′-sulfamoylbiphenyl-4-yl)acetamide,

N-(3-N-(4-methoxyphenylthiocarbonyl)amidinobenzyl)-N-cyclopentyl-2-(2′-sulfamoylbiphenyl-4-yl)acetamide,

N-(3-N-(4-methoxyphenylthiocarbonyl)amidinobenzyl)-N-benzyl-2-(2′-sulfamoylbiphenyl-4-yl)acetamide,

N-(3-N-(4-methoxyphenylthiocarbonyl)amidinobenzyl)-N-phenyl-2-(2′-sulfamoylbiphenyl-4-yl)acetamide.

Reaction of the “amidino compounds” with 1-acetoxyethyl-4-nitrophenyl carbonate gives the following

N-(3-N-acetoxyethoxycarbonylamidinobenzyl)-N-propyl-2-(2′-sulfamoylbiphenyl-4-yl)acetamide,

N-(3-N-acetoxyethoxycarbonylamidinobenzyl)-N-methyl-2-(2′-sulfamoylbiphenyl-4-yl)acetamide,

N-(3-N-acetoxyethoxycarbonylamidinobenzyl)-N-ethyl-2-(2′-sulfamoylbiphenyl-4-yl)acetamide,

N-(3-N-acetoxyethoxycarbonylamidinobenzyl)-N-isopropyl-2-(2′-sulfamoylbiphenyl-4-yl)acetamide,

N-(3-N-acetoxyethoxycarbonylamidinobenzyl)-N-butyl-2-(2′-sulfamoylbiphenyl-4-yl)acetamide,

N-(3-N-acetoxyethoxycarbonylamidinobenzyl)-N-isobutyl-2-(2′-sulfamoylbiphenyl-4-yl)acetamide,

N-(3-N-acetoxyethoxycarbonylamidinobenzyl)-N-pentyl-2-(2′-sulfamoylbiphenyl-4-yl)acetamide,

N-(3-N-acetoxyethoxycarbonylamidinobenzyl)-N-sec-butyl-2-(2′-sulfamoylbiphenyl-4-yl)acetamide,

N-(3-N-acetoxyethoxycarbonylamidinobenzyl)-N-cyclohexylmethyl-2-(2′-sulfamoylbiphenyl-4-yl)acetamide,

N-(3-N-acetoxyethoxycarbonylamidinobenzyl)-N-cyclohexyl-2-(2′-sulfamoylbiphenyl-4-yl)acetamide,

N-(3-N-acetoxyethoxycarbonylamidinobenzyl)-N-cyclopentyl-2-(2′-sulfamoylbiphenyl-4-yl)acetamide,

N-(3-N-acetoxyethoxycarbonylamidinobenzyl)-N-benzyl-2-(2′-sulfamoylbiphenyl-4-yl)acetamide,

N-(3-N-acetoxyethoxycarbonylamidinobenzyl)-N-phenyl-2-(2′-sulfamoylbiphenyl-4-yl)acetamide.

### EXAMPLE 6

The reaction is carried out analogously to S. M. Rahmathullah et al. in J. Med. Chem. 1999, 42, 3994-4000.

Reaction of ethyl chloroformate and the following “N-hydroxyamidino compounds”

N-(3-N-hydroxyamidinobenzyl)-N-propyl-2-(2′-sulfamoylbiphenyl-4-yl)acetamide,

N-(3-N-hydroxyamidinobenzyl)-N-methyl-2-(2′-sulfamoylbiphenyl-4-yl)acetamide,

N-(3-N-hydroxyamidinobenzyl)-N-ethyl-2-(2′-sulfamoylbiphenyl-4-yl)acetamide,

N-(3-N-hydroxyamidinobenzyl)-N-isopropyl-2-(2′-sulfamoylbiphenyl-4-yl)acetamide,

N-(3-N-hydroxyamidinobenzyl)-N-butyl-2-(2′-sulfamoylbiphenyl-4-yl)acetamide,

N-(3-N-hydroxyamidinobenzyl)-N-isobutyl-2-(2′-sulfamoylbiphenyl-4-yl)acetamide,

N-(3-N-hydroxyamidinobenzyl)-N-pentyl-2-(2′-sulfamoylbiphenyl-4-yl)acetamide,

N-(3-N-hydroxyamidinobenzyl)-N-sec-butyl-2-(2′-sulfamoylbiphenyl-4-yl)acetamide,

N-(3-N-hydroxyamidinobenzyl)-N-cyclohexylmethyl-2-(2′-sulfamoylbiphenyl-4-yl)acetamide,

N-(3-N-hydroxyamidinobenzyl)-N-cyclohexyl-2-(2′-sulfamoylbiphenyl-4-yl)acetamide,

N-(3-N-hydroxyamidinobenzyl)-N-cyclopentyl-2-(2′-sulfamoylbiphenyl-4-yl)acetamide,

N-(3-N-hydroxyamidinobenzyl)-N-benzyl-2-(2′-sulfamoylbiphenyl-4-yl)acetamide,

N-(3-N-hydroxyamidinobenzyl)-N-phenyl-2-(2′-sulfamoylbiphenyl-4-yl)acetamide,

gives the following

N-(3-N-ethoxycarbonyloxyamidinobenzyl)-N-propyl-2-(2′-sulfamoylbiphenyl-4-yl)acetamide,

N-(3-N-ethoxycarbonyloxyamidinobenzyl)-N-methyl-2-(2′-sulfamoylbiphenyl-4-yl)acetamide,

N-(3-N-ethoxycarbonyloxyamidinobenzyl)-N-ethyl-2-(2′-sulfamoylbiphenyl-4-yl)acetamide,

N-(3-N-ethoxycarbonyloxyamidinobenzyl)-N-isopropyl-2-(2′-sulfamoylbiphenyl-4-yl)acetamide,

N-(3-N-ethoxycarbonyloxyamidinobenzyl)-N-butyl-2-(2′-sulfamoylbiphenyl-4-yl)acetamide,

N-(3-N-ethoxycarbonyloxyamidinobenzyl)-N-isobutyl-2-(2′-sulfamoylbiphenyl-4-yl)acetamide,

N-(3-N-ethoxycarbonyloxyamidinobenzyl)-N-pentyl-2-(2′-sulfamoylbiphenyl-4-yl)acetamide,

N-(3-N-ethoxycarbonyloxyamidinobenzyl)-N-sec-butyl-2-(2′-sulfamoylbiphenyl-4-yl)acetamide,

N-(3-N-ethoxycarbonyloxyamidinobenzyl)-N-cyclohexylmethyl-2-(2′-sulfamoylbiphenyl-4-yl)acetamide,

N-(3-N-ethoxycarbonyloxyamidinobenzyl)-N-cyclohexyl-2-(2′-sulfamoylbiphenyl-4-yl)acetamide,

N-(3-N-ethoxycarbonyloxyamidinobenzyl)-N-cyclopentyl-2-(2′-sulfamoylbiphenyl-4-yl)acetamide,

N-(3-N-ethoxycarbonyloxyamidinobenzyl)-N-benzyl-2-(2′-sulfamoylbiphenyl-4-yl)acetamide,

N-(3-N-ethoxycarbonyloxyamidinobenzyl)-N-phenyl-2-(2′-sulfamoylbiphenyl-4-yl)acetamide.

### EXAMPLE 7

Analogously to Example 5, the following compounds are obtained

N-(3-N-(N,N-diethylaminoethoxycarbonyl)amidinobenzyl)-N-propyl-2-(2′-sulfamoylbiphenyl-4-yl)acetamide,

N-(3-N-(N,N-diethylaminoethoxycarbonyl)amidinobenzyl)-N-methyl-2-(2′-sulfamoylbiphenyl-4-yl)acetamide,

N-(3-N-(N,N-diethylaminoethoxycarbonyl)amidinobenzyl)-N-ethyl-2-(2′-sulfamoylbiphenyl-4-yl)acetamide,

N-(3-N-(N,N-diethylaminoethoxycarbonyl)amidinobenzyl)-N-isopropyl-2-(2′-sulfamoylbiphenyl-4-yl)acetamide,

N-(3-N-(N,N-diethylaminoethoxycarbonyl)amidinobenzyl)-N-butyl-2-(2′-sulfamoylbiphenyl-4-yl)acetamide,

N-(3-N-(N,N-diethylaminoethoxycarbonyl)amidinobenzyl)-N-isobutyl-2-(2′-sulfamoylbiphenyl-4-yl)acetamide,

N-(3-N-(N,N-diethylaminoethoxycarbonyl)amidinobenzyl)-N-pentyl-2-(2′-sulfamoylbiphenyl-4-yl)acetamide,

N-(3-N-(N,N-diethylaminoethoxycarbonyl)amidinobenzyl)-N-sec-butyl-2-(2′-sulfamoylbiphenyl-4-yl)acetamide,

N-(3-N-(N,N-diethylaminoethoxycarbonyl)amidinobenzyl)-N-cyclohexylmethyl-2-(2′-sulfamoylbiphenyl-4-yl)acetamide,

N-(3-N-(N,N-diethylaminoethoxycarbonyl)amidinobenzyl)-N-cyclohexyl-2-(2′-sulfamoylbiphenyl-4-yl)acetamide,

N-(3-N-(N,N-diethylaminoethoxycarbonyl)amidinobenzyl)-N-cyclopentyl-2-(2′-sulfamoylbiphenyl-4-yl)acetamide,

N-(3-N-(N,N-diethylaminoethoxycarbonyl)amidinobenzyl)-N-benzyl-2-(2′-sulfamoylbiphenyl-4-yl)acetamide,

N-(3-N-(N,N-diethylaminoethoxycarbonyl)amidinobenzyl)-N-phenyl-2-(2′-sulfamoylbiphenyl-4-yl)acetamide,

N-(3-N-(N-methylpiperidin-4-yloxycarbonyl)amidinobenzyl)-N-propyl-2-(2′-sulfamoylbiphenyl-4-yl)acetamide,

N-(3-N-(N-methylpiperidin-4-yloxycarbonyl)amidinobenzyl)-N-methyl-2-(2′-sulfamoylbiphenyl-4-yl)acetamide,

N-(3-N-(N-methylpiperidin-4-yloxycarbonyl)amidinobenzyl)-N-ethyl-2-(2′-sulfamoylbiphenyl-4-yl)acetamide,

N-(3-N-(N-methylpiperidin-4-yloxycarbonyl)amidinobenzyl)-N-isopropyl-2-(2′-sulfamoylbiphenyl-4-yl)acetamide,

N-(3-N-(N-methylpiperidin-4-yloxycarbonyl)amidinobenzyl)-N-butyl-2-(2′-sulfamoylbiphenyl-4-yl)acetamide,

N-(3-N-(N-methylpiperidin-4-yloxycarbonyl)amidinobenzyl)-N-isobutyl-2-(2′-sulfamoylbiphenyl-4-yl)acetamide,

N-(3-N-(N-methylpiperidin-4-yloxycarbonyl)amidinobenzyl)-N-pentyl-2-(2′-sulfamoylbiphenyl-4-yl)acetamide,

N-(3-N-(N-methylpiperidin-4-yloxycarbonyl)amidinobenzyl)-N-sec-butyl-2-(2′-sulfamoylbiphenyl-4-yl)acetamide,

N-(3-N-(N-methylpiperidin-4-yloxycarbonyl)amidinobenzyl)-N-cyclohexylmethyl-2-(2′-sulfamoylbiphenyl-4-yl)acetamide,

N-(3-N-(N-methylpiperidin-4-yloxycarbonyl)amidinobenzyl)-N-cyclohexyl-2-(2′-sulfamoylbiphenyl-4-yl)acetamide,

N-(3-N-(N-methylpiperidin-4-yloxycarbonyl)amidinobenzyl)-N-cyclopentyl-2-(2′-sulfamoylbiphenyl-4-yl)acetamide,

N-(3-N-(N-methylpiperidin-4-yloxycarbonyl)amidinobenzyl)-N-benzyl-2-(2′-sulfamoylbiphenyl-4-yl)acetamide,

N-(3-N-(N-methylpiperidin-4-yloxycarbonyl)amidinobenzyl)-N-phenyl-2-(2′-sulfamoylbiphenyl-4-yl)acetamide,

N-(3-N-(pyridin-2-ylethoxycarbonyl)amidinobenzyl)-N-propyl-2-(2′-sulfamoylbiphenyl-4-yl)acetamide,

N-(3-N-(pyridin-2-ylethoxycarbonyl)amidinobenzyl)-N-methyl-2-(2′-sulfamoylbiphenyl-4-yl)acetamide,

N-(3-N-(pyridin-2-ylethoxycarbonyl)amidinobenzyl)-N-ethyl-2-(2′-sulfamoylbiphenyl-4-yl)acetamide,

N-(3-N-(pyridin-2-ylethoxycarbonyl)amidinobenzyl)-N-isopropyl-2-(2′-sulfamoylbiphenyl-4-yl)acetamide,

N-(3-N-(pyridin-2-ylethoxycarbonyl)amidinobenzyl)-N-butyl-2-(2′-sulfamoylbiphenyl-4-yl)acetamide,

N-(3-N-(pyridin-2-ylethoxycarbonyl)amidinobenzyl)-N-isobutyl-2-(2′-sulfamoylbiphenyl-4-yl)acetamide,

N-(3-N-(pyridin-2-ylethoxycarbonyl)amidinobenzyl)-N-pentyl-2-(2′-sulfamoylbiphenyl-4-yl)acetamide,

N-(3-N-(pyridin-2-ylethoxycarbonyl)amidinobenzyl)-N-sec-butyl-2-(2′-sulfamoylbiphenyl-4-yl)acetamide,

N-(3-N-(pyridin-2-ylethoxycarbonyl)amidinobenzyl)-N-cyclohexylmethyl-2-(2′-sulfamoylbiphenyl-4-yl)acetamide,

N-(3-N-(pyridin-2-ylethoxycarbonyl)amidinobenzyl)-N-cyclohexyl-2-(2′-sulfamoylbiphenyl-4-yl)acetamide,

N-(3-N-(pyridin-2-ylethoxycarbonyl)amidinobenzyl)-N-cyclopentyl-2-(2′-sulfamoylbiphenyl-4-yl)acetamide,

N-(3-N-(pyridin-2-ylethoxycarbonyl)amidinobenzyl)-N-benzyl-2-(2′-sulfamoylbiphenyl-4-yl)acetamide,

N-(3-N-(pyridin-2-ylethoxycarbonyl)amidinobenzyl)-N-phenyl-2-(2′-sulfamoylbiphenyl-4-yl)acetamide.

### EXAMPLE 8

Reaction of 2,2,2-trifluoroacetamide with ethyl bromoacetate analogously to 1.1 and further reaction analogously to 1.2, 1.3, 3.1, 3.2 and 3.3 gives N-(3-amidinobenzyl)-2-(2′-sulfamoylbiphenyl-4-yl)-N-ethoxycarbonylmethylacetamide.

Analogous reaction with methyl bromopropionate gives the compound N-(3-amidinobenzyl)-2-(2′-sulfamoylbiphenyl-4-yl)-N-methoxycarbonylethylacetamide.

### EXAMPLE 9

Preparation of N-(3-amidinobenzyl)-2-(2′-sulfamoylbiphenyl-4-yl)-N-(1-methyltetrazol-5-ylethyl)acetamide (“GA”):

Analogously to the above examples, the use of 3-bromopropionitrile gives the compound N-(3-(5-methyl-1,2,4-oxadiazol-3-yl)benzyl)-2-(2′-sulfamoylbiphenyl-4-yl)-N-(2-cyanoethyl)acetamide.

The conversion of the cyano group into the 1H-tetrazol-5-yl group is carried out by conventional methods by reaction with sodium azide or trimethylsilyl azide, giving N-(3-(5-methyl-1,2,4-oxadiazol-3-yl)benzyl)-2-(2′-sulfamoylbiphenyl-4-yl)-N-(2-(1H-tetrazol-5-yl)ethyl)acetamide.

Methylation using methyl iodide and subsequent hydrogenation in methanol/acetic acid with Raney nickel catalysis gives the compound “GA” after removal of the catalyst and, conventional work-up.

Analogous reaction of

2-methoxyethyl bromide,

1-bromodimethyl ether and

4-methoxybutyl bromide

gives the following compounds

N-(3-amidinobenzyl)-2-(2′-sulfamoylbiphenyl-4-yl)-N-methoxyethylacetamide,

N-(3-amidinobenzyl)-2-(2′-sulfamoylbiphenyl-4-yl)-N-methoxymethylacetamide,

N-(3-amidinobenzyl)-2-(2′-sulfamoylbiphenyl-4-yl)-N-methoxybutylacetamide.

The examples below relate to pharmaceutical preparations:

### Example A

Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### Example B

Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C

Solution

A solution is prepared from 1 g of an active ingredient of the formula I, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D

Ointment

500 mg of an active ingredient of the formula I are mixed with 99.5 g of Vaseline under aseptic conditions.

### Example E

Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

Coated Tablets

Tablets are pressed analogously to Example E and subsequently coated in a conventional manner with a coating of sucrose, potato starch, talc, tragacanth and dye.

### Example G

Capsules

2 kg of active ingredient of the formula I are introduced into hard gelatine capsules in a conventional manner in such a way that each capsule contains 20 mg of the active ingredient.

### Example H

Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 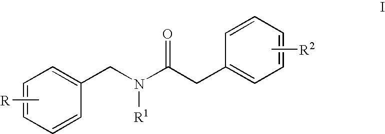
in which
R is CH₂NH₂, —CO—N═C(NH₂)₂, —NH—C(═NH)—NH₂ or —C(═NH)—NH₂, each of which may also be monosubstituted by OH, —OCOOA, —OCOO(CH₂)ₙNAA′, —COO(CH₂)ₙNAA′, —OCOO(CH₂)ₘ-Het, —COO(CH₂)ₘ-Het, —CO—CAA′—R³, —COO—CAA′—R³, COOA, COSA, COOAr, COOAr or by a conventional amino-protecting group, or is 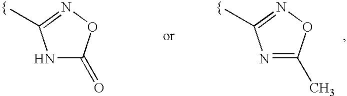
R¹ is unbranched, branched or cyclic alkyl having 1-20 carbon atoms, in which one or two CH₂ groups can be replaced by O or S atoms, or is Ar, Ar′ or X,
R² is phenyl which is monosubstituted by S(O)ₚA, S(O)ₚNHA, CF₃, COOA, CH₂NHA, CN or OA,
R³ is —C(Hal)₃, —O(C═O)A or 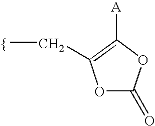
Ar is phenyl or naphthyl, each of which is unsubstituted or monosubstituted, disubstituted or trisubstituted by A, OA, NAA′, NO₂, CF₃, CN, Hal, NHCOA, COOA, CONAA′, S(O)ₚA or S(O)ₚNAA′,
Ar′ is —(CH₂)ₙ—Ar,
A is H or unbranched, branched or cyclic alkyl having 1-20 carbon atoms,
A′ is unbranched, branched or cyclic alkyl having 1-10 carbon atoms,
Het is a monocyclic or bicyclic saturated, unsaturated or aromatic heterocyclic radical having from 1 to 4 N, O and/or S atoms, bonded via N or C, which may be unsubstituted or substituted by A,
X is —(CH₂)ₙ—Y,
Y is COOA or 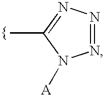
Hal is F, Cl, Br or I,
m is 0 or 1,
n is 1, 2, 3, 4, 5 or 6,
p is 0, or 2,
and pharmaceutically tolerated salts, solvates and stereoisomers thereof.

2. Compounds according to claim 1, in which
R is —C(═NH)—NH₂, which may also be monosubstituted by OH or a conventional amino-protecting group, or is 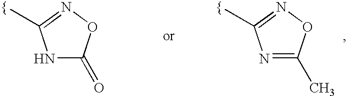
and pharmaceutically tolerated salts, solvates and stereoisomers thereof.

3. Compounds according to claim 1, in which
R is —C(═NH)—NH₂, which may also be monosubstituted by OH or a conventional amino-protecting group, or is 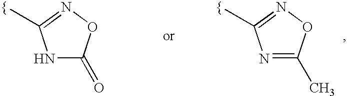
R¹ is unbranched, branched or cyclic alkyl having 1-8 carbon atoms, in which one CH₂ group may be replaced by O, or is Ar, Ar′ or X,
and pharmaceutically tolerated salts, solvates and stereoisomers thereof.

4. Compounds according to claim 1, in which
R is —C(═NH)—NH₂, which may also be monosubstituted by OH or a conventional amino-protecting group, or is 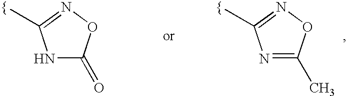
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
and pharmaceutically tolerated salts, solvates and stereoisomers thereof.

5. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 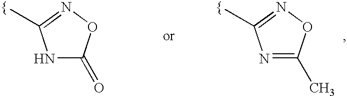
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA,CN or OA,
R³ is —CCl₃ or —O(C═O)A,
and pharmaceutically tolerated salts, solvates and stereoisomers thereof.

6. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 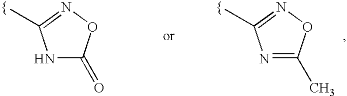
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
and pharmaceutically tolerated salts, solvates and stereoisomers thereof.

7. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 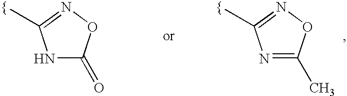
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
and pharmaceutically tolerated salts, solvates and stereoisomers thereof.

8. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 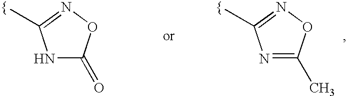
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
A and A′ are each, independently of one another, H or unbranched, branched or cyclic alkyl having 1-8 carbon atoms,
and pharmaceutically tolerated salts, solvates and stereoisomers thereof.

9. Compounds according to claim 1, in which
R is —NH—C(═NH)—NH₂, —CO—N═C(NH₂)₂, —C(═NH)—NH₂, which may also be monosubstituted by OH, O—COA, O—COAr, OCOOA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 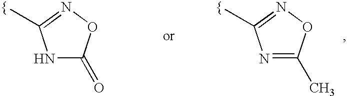
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
Het is a monocyclic saturated or aromatic heterocyclic radical having from 1 to 2 N and/or O atoms,
and pharmaceutically tolerated salts, solvates and stereoisomers thereof.

10. Compounds according to claim 1, in which
R is CH₂NH₂, CH₂NHCOA or CH₂NHCOOA, —C(═NH)—NH₂, which may also be monosubstituted by OH, O—COA, O—COAr, OCOGA, OCOO(CH₂)ₙN(A)₂, COO(CH₂)ₙN(A)₂, OCOO(CH₂)ₘHet, COO—(CH₂)ₘ-Het, CO—C(A)₂—R³, COOA, COSA, COSAr, COOAr, COOAr′, COA, COAr, COAr′ or by a conventional amino-protecting group, or is 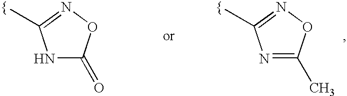
R¹ is unbranched, branched or cyclic alkyl having 1-8 carbon atoms, in which one CH₂ group may be replaced by O, or is Ar, Ar′ or X,
R² is phenyl which is monosubstituted by SA, SOA, SO₂A, SO₂NHA, CF₃, COOA, CH₂NHA, CN or OA,
R³ is —CCl₃ or —O(C═O)A,
Ar is phenyl which is unsubstituted or monosubstituted by A, OA, CF₃, Hal or SO₂NH₂,
Ar′ is benzyl which is unsubstituted or monosubstituted, disubstituted or trisubstituted by fluorine,
Het is a monocyclic saturated or aromatic heterocyclic radical having from 1 to 2 N and/or O atoms,
and pharmaceutically tolerated salts, solvates and stereoisomers thereof.

11. Compounds according to claim 1
a) N-(3-amidinobenzyl)-N-propyl-2-(2′-aminosulfonylbiphenyl-4-yl)acetamide,
b) N-(3-amidinobenzyl)-N-propyl-2-(2′-methylsulfonylbiphenyl-4-yl)acetamide,
c) N-(3-amidinobenzyl)-N-phenyl-2-(2′-sulfamoylbiphenyl-4-yl)acetamide,
and pharmaceutically tolerated salts and solvates thereof.

12. Process for the preparation of compounds of the formula I according to claim 1 in which R is amidino, and salts thereof, characterised in that
a) they are liberated from one of their functional derivatives by treatment with a solvolysing or hydrogenolysing agent,
and/or
b) a base or acid of the formula I is converted into one of its salts.

13. Compounds of the formula I according to claims 1 to 11 and physiologically acceptable salts and solvates thereof as medicaments.

14. Medicaments according to claim 13 as inhibitors of coagulation factor Xa.

15. Medicaments according to claim 13 as inhibitors of coagulation factor VIIa.

16. Medicaments according to claim 13, 14 or 15 for the treatment of thromboses, myocardial infarction, arteriosclerosis, inflammation, apoplexia, angina pectoris, restenosis after angioplasty, claudicatio intermittens, tumours, tumour diseases and/or tumour metastases.

17. Pharmaceutical preparation comprising at least one medicament according to one of claims 13 to 16 and, if desired, excipients and/or adjuvants and, if desired, other active ingredients.

18. Use of compounds according to claims 1 to 11 and/or physiologically acceptable salts and solvates thereof for the preparation of a medicament for the treatment of thromboses, myocardial infarction, arteriosclerosis, inflammation, apoplexia, angina pectoris, restenosis after angioplasty, claudicatio intermittens, tumours, tumour diseases and/or tumour metastases.
